(19) United States
(12) Patent Application Publication
(10) Pub. No.: US 2013/0281400 A1
(43) Pub. Date: Oct. 24, 2013

(54) Metabolic Downregulation for Cell Survival

(71) Applicants: James J. Yoo, Winston Salem, NC (US); Sang Jin Lee, Winston Salem, NC (US); Jaehyun Kim, Winston Salem, NC (US); Anthony Atala, Winston Salem, NC (US)

(72) Inventors: James J. Yoo, Winston Salem, NC (US); Sang Jin Lee, Winston Salem, NC (US); Jaehyun Kim, Winston Salem, NC (US); Anthony Atala, Winston Salem, NC (US)

(73) Assignee: Wake Forest University Health Sciences, Winston Salem, NC (US)

(21) Appl. No.: 13/882,433

(22) PCT Filed: Nov. 10, 2011
(86) PCT No.: PCT/US11/60196

§ 371 Date: Jul. 9, 2013

Related U.S. Application Data
(60) Provisional application No. 61/412,201, filed on Nov. 10, 2010.

(51) Int. Cl. C12N 5/071

(52) U.S. Cl. CPC C12N 5/0658
USPC 514/46; 435/375; 435/354; 435/366

(57) ABSTRACT

The present provides a system and method of maintaining and/or increasing cell viability by downregulating cellular metabolic rate under hypoxic conditions. The present invention also relates to a system and method of prolonging the survival of implanted cells that are under hypoxic condition until host neovascularization is achieved.

### BACKGROUND OF THE INVENTION

Building a clinically relevant sized tissue or organ using cells requires maintenance of viable cells until host vasculature is established and integrated into the implanted engineered constructs. Tissue engineering (TE) generally includes use of a scaffold that provides an architecture on which seeded cells are matured into tissues and organs. One of the foremost challenges in TE is the limitation imposed on oxygen supply immediately following Implantation of the cell-scaffold construct. Supplying sufficient oxygen to the engineered tissue is essential for survival and integration of transplanted cells. Unfortunately, the lack of vascularization of implanted tissues and inadequate removal of waste products prevents diffusion of oxygen into the interior of the scaffold. This makes the survival rate of the seeded cells very low, and in many instances, only the cells located near the surface of implant survive. Such limitations have led to a general conception that cell or tissue components may not be implanted in large volumes, as the delay in vasculogenesis often results in premature cell death due to the inadequate supply of oxygen and nutrients.

Oxygen diffusion is of crucial importance especially when building a clinically relevant sized tissue or organ. The distance that oxygen must diffuse between capillary lumen and a cell membrane is almost never more than 40 to 200 μm (Chow et al., 2001, Biophys J, 81(2):685-96; Chow et al., 2001, Biophys J, 81(2):675-84) whereas, in most clinical grafts, the distance for oxygen from the edge of the graft to the center of the graft is a minimum 5 mm, or approximately fifty times the normal diffusion distance (Muschler et al., 2004, J Bone Joint Surg Am, 86-A(7):1541-58). In this setting, diffusion is able to support only a limited number of transplanted cells, and this creates the center of the graft where oxygen tension is too low to support viable cells, resulting in central necrosis. This is a major reason why many cell transplantation methods work very well in small animals but fail in larger animals and humans. Currently, oxygen diffusion has been limiting the engineering of large functional tissue implants for human application.

Several methods have been developed to overcome this challenge. For example, strategies including the use of oxygen rich fluids such as perfluorocarbons and silicone oils (Radisic et al., 2006, Tissue Eng, 12(8):2077-91; Leung et al., 1997, J Chem Technol Biotechnology, 68:37-46), the use of angiogenic factors, such as vascular endothelial growth factors (VEGF) and endothelial cells, and cell-support matrices that permit enhanced diffusion across the entire implant (De Coppi et al., 2005, Tissue Eng, 11(7-8):1034-44; Kaigler et al., 2006, J Bone Miner Res, 21(5):735-44; Nomi et al., 2002, Mol Aspects Med, 23(6):463-83) have all been attempted. However, none of these strategies have been successful to date in achieving survival of a clinically applicable large tissue mass (Harrison et al., 2007, Biomaterials, 28(31):4628-34). Although these measures are designed to facilitate the delivery of oxygen, they are unable to reduce the oxygen demand of the cells.

One potential solution is to develop methods to maintain cell viability over a long-term by downregulating cellular metabolism until host vascularization is established. Adenosine, a nucleoside which functions as an energy transferring molecule, is reported to increase during hypoxia and functions as a modulator of ion-channel arrest (Buck, 2004, Comp Biochem Physiol B Biochem Mol Biol 139(3):401-414). This results in a decrease in ATP consumption and thus, oxygen demand. Thus, a need exists for a method of promoting cell survival under hypoxic conditions by exploiting this property of adenosine. The present invention satisfies this need.

### SUMMARY OF THE INVENTION

The invention provides a method of increasing the viability of a cell under a hypoxic condition. In one embodiment, the invention comprises contacting the cell with an effective amount of adenosine to reduce the oxygen demand of the cell.

In one embodiment, the effective amount of adenosine downregulates the metabolic rate of the cell.

In one embodiment, contacting the cell with an effective amount of adenosine further results in a steady state of cellular metabolic activity.

In one embodiment, the cell resumes a normal proliferation rate when the adenosine is removed from the cell.

In one embodiment, the effective amount of adenosine is greater than about 1 mM. In another embodiment, the amount of adenosine is between about 1 mM and about 10 mM.

In one embodiment, the cell is a myoblast. In another embodiment, the cell is a murine myoblast. In yet another embodiment, the cell is a human myoblast.

The invention also provides a method of increasing cellular survival in a tissue-engineered construct during vasculogenesis. In one embodiment, the method comprises administering an effective amount of adenosine to the cells in the tissue-engineered construct to downregulate the metabolic rate of the cells until host vascularization is established.

In one embodiment, the effective amount of adenosine is greater than about 1 mM. In another embodiment, the effective amount of adenosine is between about 1 mM and about 10 mM.

The invention also provides a method of prolonging the survival of an implanted cell that is under a hypoxic condition in a host. In one embodiment, the method comprises contacting the cell with an effective amount of adenosine to reduce the oxygen demand of the cell until host neovascularization is achieved.

In one embodiment, the effective amount of adenosine downregulates the metabolic rate of the cell.

In one embodiment, contacting the cell with an effective amount of adenosine further results in a steady state of cellular metabolic activity.

In one embodiment, the hypoxic cell resumes a normal proliferation rate when the effects of adenosine are removed.

In one embodiment, the effective amount of adenosine is greater than about 1 mM. In another embodiment, the amount of adenosine is between about 1 mM and about 10 mM.

In one embodiment, the cell is a myoblast. In another embodiment, the cell is a murine myoblast. In yet another embodiment, the cell is a human myoblast.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a chart depicting the targeted effect of adenosine on cellular activity with respect to time.

FIG. 2 is a chart depicting the effect of adenosine on C2C12 metabolic activity under either normoxic or hypoxic condition.

FIG. 3 is a chart depicting the effect of adenosine dose on cellular metabolic activity under hypoxic condition throughout.

FIG. 4 is a chart depicting the long term effect of adenosine on restoring cellular activity.

### DETAILED DESCRIPTION OF THE INVENTION

The present invention is directed towards a system and method of maintaining and/or increasing cell viability by downregulating cellular metabolic rate under hypoxic conditions. This concept also represents a novel method for increasing cellular survival in tissue-engineered constructs during vasculogenesis, whereby cell viability is increased by downregulating cellular metabolic rate until host vascularization is established. The present invention also relates to a system and method of prolonging the survival of implanted cells that are under hypoxic condition until host neovascularization is achieved.

### Definitions:

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used herein, the term “treatment” or “treating” is defined as the application or administration of a therapeutic agent, i.e., a compound, such as adenosine, useful within the invention (alone or in combination with another agent), to a subject, or application or administration of a therapeutic agent to an isolated tissue or cell either engineered or from a subject (e.g., for diagnosis or ex vivo applications), with the purpose to cure, heal, alleviate, relieve, alter, remedy, ameliorate, improve or affect the condition being treated.

As used herein, the term “patient” or “subject” refers to a human or a non-human animal. Non-human animals include, for example, livestock and pets, such as ovine, bovine, porcine, canine, feline and murine mammals. Preferably, the patient or subject is human.

As used herein, the terms “effective amount,” “pharmaceutically effective amount” and “therapeutically effective amount” refer to a non-toxic but sufficient amount of an agent to provide the desired biological result. That result can be reduction and/or alleviation of the signs, symptoms, or causes of a disease, or any other desired alteration of a biological system. An appropriate therapeutic amount in any individual case may be determined by one of ordinary skill in the art using routine experimentation.

### Methods of the Invention

As demonstrated herein, cells (such as murine myoblasts and human myoblasts, for example) under hypoxic condition maintain a steady state of metabolic activity when treated with adenosine. Hypoxic cells not treated with adenosine proceed to die. Hypoxic cells can resume their normal proliferation rate when the effects of adenosine are removed. Thus, the present invention is directed towards a system and method of maintaining and/or increasing cell viability by downregulating cellular metabolic rate under hypoxic conditions. In one embodiment of the present invention, the method is performed by applying adenosine to cells under hypoxic conditions and prolonging cell survival by decreasing the metabolic activity to a steady hypometabolic state, thus reducing O₂ demand.

The present invention is also directed towards a system and method of increasing cellular survival in tissue-engineered constructs during vasculogenesis. In one embodiment, the method is performed by applying adenosine to cells to downregulate cellular metabolic rate until host vascularization is established.

The present invention further relates to a system and method of prolonging the survival of implanted cells that are under hypoxic condition by adding adenosine to cells until host neovascularization is achieved.

The methods of the present invention are markedly different than existing methods, in that the present invention works by lowering the oxygen demand of the cells by downregulating their metabolic rate to a hypometabolic steady state, instead of focusing on preventing the extreme hypoxia that immediately follows implantation of cells to the scaffold by facilitating the delivery of oxygen.

The present invention can be incorporated into the use of biomaterials for medical implants, devices, scaffolds, etc. The present invention may also be used to supplement cell culture media for controlled cell growth. Additionally, the present invention may be used with various formulations administered as an injection, ointment, dressing or spray, for example, to treat ischemia and trauma.

As contemplated herein, the present invention may be used in conjunction with the engineering of clinically relevant tissues for functional recovery, tissue and organ salvage due to trauma and ischemia of various tissues and organs, organ transplantation and reconstructive procedures involving tissue flap and grafts (intra and post-operative supplements). For example, the present invention is vital for extending the viability of larger engineered constructs seeded with higher densities of cells in vivo. With vascularization of tissue scaffolds estimated at 0.5-1 mm/day in tissue, maintaining cell viability in the middle of a tissue scaffold for 10 days permits the use of centimeter sized tissue scaffolds (Cao et al., 2006, Biomaterials, 27(14):2854-64).

In instances where severe oxygen limitation is present, cell death occurs when ATP production fails to meet the energetic maintenance demands of ionic and osmotic equilibrium. The decline of high energy phosphates level leads to a failure of ion-motive ATPases, followed by membrane depolarization, which leads to uncontrolled cellular swelling and, ultimately, to cell necrosis. Ion-motive ATPase is one of the dominant energy-consuming processes of cells at standard metabolic rate (19-28%). Under cellular stress, priority of energy consumption even shifts from protein synthesis to more critical cell function involved in osmotic and ionic homeostasis (20-80%). One of the mechanisms is the passive ion channel arrest, resulting in decreases in membrane permeability (“ion channel arrest”) that dramatically reduce the energetic costs of ion-balancing ATPases.

### Addition of Adenosine

Adenosine, a nucleoside known for its function as an energy transferring molecule, is also known to function as a modulator of hypoxia-induced ion-channel arrest, which eventually leads to lowering of ATP consumption, and thus oxygen demand. Moreover, it is also known to stimulate angiogenesis. Thus, the present invention provides for the application of adenosine to cell-seeded scaffolds under the hypoxic condition to improve cell survival rate via the downregulation of metabolic rate, and thus lowering oxygen demand and consumption. As contemplated herein, agonists to adenosine may also be used in a similar manner to lower the oxygen demand of cells and tissues. Similar results are observed when adenosine is injected into muscle tissue, demonstrating the preservation of cellular viability and tissue architecture. Adenosine may be administered alone as a composition or within a formulation, as would be understood by those skilled in the art.

In another aspect of the present invention, the effects of adenosine on either cellular metabolic activity or proliferation are dose dependent. In one embodiment of the present invention, the effective concentration of adenosine to be administered to the cells is greater than about 1 mM. In another embodiment, the effective concentration of adenosine is between about 1 mM to about 10 mM and any and all whole or partial increments therebetween, including about 2 mM, about 3 mM, about 4 mM, about 5 mM, about 6 mM, about 7 mM, about 8 mM, about 9 mM, and about 10 mM. As the dose of adenosine increases from 1 to 10 mM, an escalation of steady hypometabolic state can be maintained under hypoxic conditions, such that the cells are able to resume their normal metabolic activity after a period of time, such as after 7 days.

The regimen of administration may affect what constitutes an effective amount. Therapeutic formulations may be administered to the cells either prior to or after a determination of cellular hypoxic levels. Further, several divided dosages, as well as staggered dosages may be administered daily or sequentially, or the dose may be continuously infused, or may be a bolus injection. Further, the dosages of any therapeutic formulations may be proportionally increased or decreased as indicated by the exigencies of the therapeutic or prophylactic situation.

Administration of adenosine to a cell may be carried out using known procedures, at dosages and for periods of time effective to treat hypoxic levels in the cell. An effective amount of adenosine necessary to achieve a therapeutic effect may vary according to factors such as the state of the disease or disorder in the cell or subject, and the ability of adenosine to treat hypoxic levels in the cell. Dosage regimens may be adjusted to provide the optimum therapeutic response. For example, several divided doses may be administered daily or the dose may be proportionally reduced as indicated by the exigencies of the therapeutic situation. One of ordinary skill in the art would be able to study the relevant factors and make the determination regarding the effective amount of adenosine without undue experimentation.

Actual dosage levels of adenosine may be varied so as to obtain an amount of adenosine that is effective to achieve the desired therapeutic response for a particular cell, composition, and mode of administration, without being toxic to the cell or subject.

In one embodiment, the compositions of the invention are formulated using one or more pharmaceutically acceptable excipients or carriers. In one embodiment, the pharmaceutical compositions of the invention comprise a therapeutically effective amount of adenosine and a pharmaceutically acceptable carrier.

Formulations including adenosine may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

### Effect of Adenosine and its Characterization on Cellular Activity

The following studies were performed to evaluate whether: 1) hypoxic cells not treated with adenosine result in necrosis; 2) cells under hypoxic condition maintain a steady state of metabolic activity when treated with adenosine; and 3) hypoxic cells resume their normal proliferation rate when the effects of adenosine is removed.

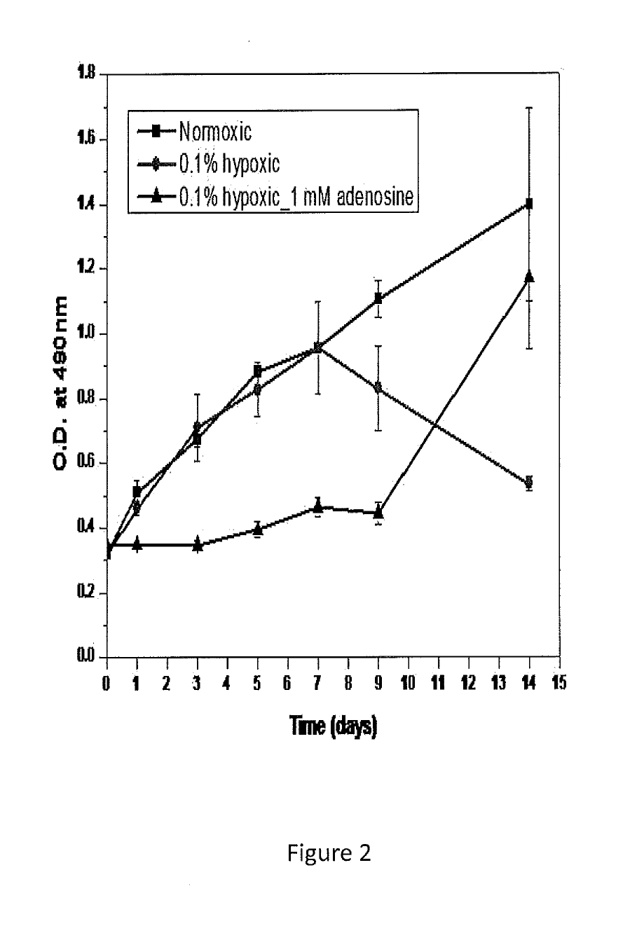

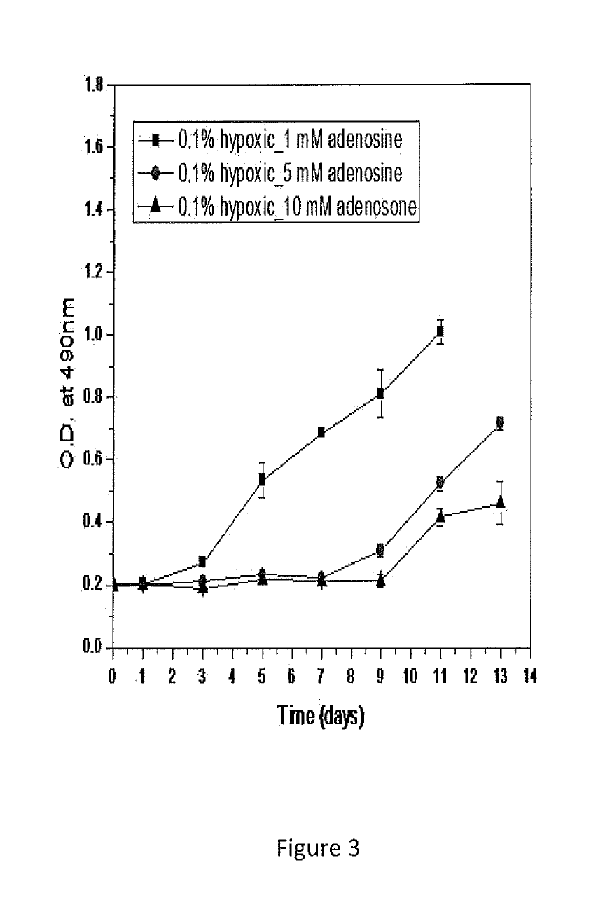

500 μL of a cell suspension (C2C12 cells, murine myoblasts) in high glucose Dulbecco's modified Eagle's medium (DMEM, Gibco) containing 10% FBS, 500 U/mL penicillin and 500 μg/mL streptomycin was placed in each well of a 48-well culture plate at a density of 1052 (FIG. 2) and 2105 (FIG. 3) cells/cm². Cells were incubated for 24 hr in normoxic conditions (21% O₂, 37° C.) prior to placement in a hypoxic chamber. At day 0, the plates designated as the hypoxic group were transferred to the hypoxic chamber (0.1% O₂). A group with no adenosine was placed under hypoxia for up to 13 days to demonstrate eventual cell death. Another group receiving daily doses of adenosine (0, 0.025, 0.25, 1, 5, and 1 mM adenosine, achieved by serial dilution) was incubated for up to 7 days under hypoxia and then placed back into normoxic conditions without additional supply of adenosine. Adenosine was refreshed by the daily exchange of media in which adenosine was completely dissolved. Media to be used under hypoxia was placed in the hypoxic chamber 24 hr prior to use for deoxygenation down to 2% O₂. The metabolic activity of viable cells at each pre-determined time point was assessed using an MTS assay, which measures mitochondrial activity of cells.

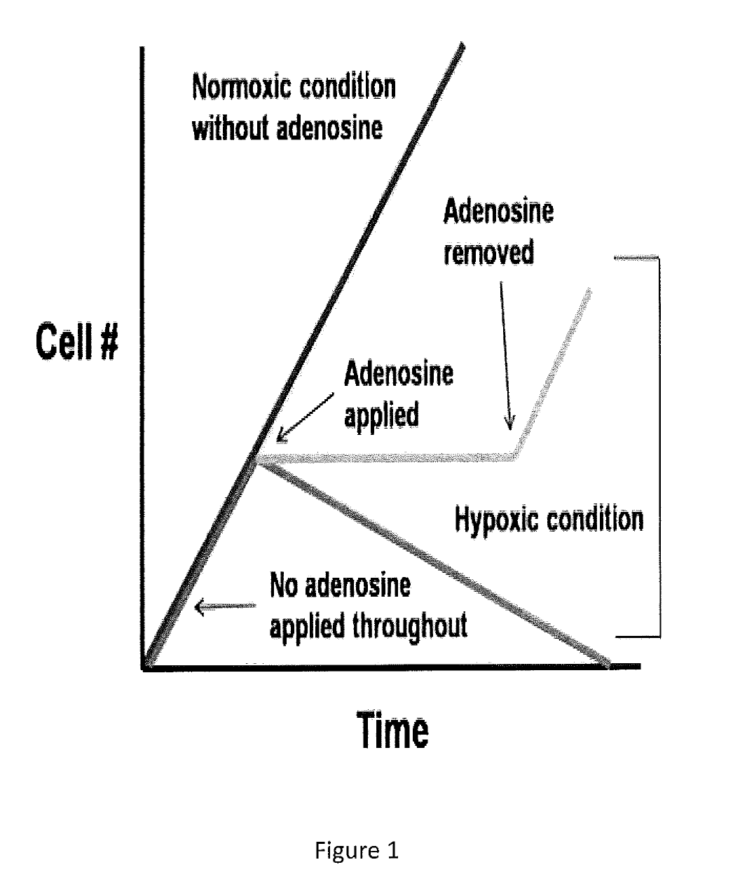

As depicted in FIG. 1, hypothetical clinical settings are simulated where seeded cells not treated with adenosine prior to host neovascularization result in cell death after implantation, but ones under the effect of adenosine maintains its viability for an extended period. Importantly, cells have to restore their normal metabolic activity and proliferation rate upon neovascularization, as the long-term maintenance of a suppressed state might not be clinically meaningful. Thus, the time period for the transition process from the hypometabolic phase to take place, and the recovered proliferation rate once the effect of adenosine is removed, are significant parameters to be evaluated.

Adenosine was used with C2C12 at passage 17, the murine myoblast cell lines, because of their relatively high metabolic activity and proliferation rate. This study demonstrated that the metabolic activity of cells grown in normoxic conditions increased linearly with respect to time (FIG. 2). Hypoxic cells not treated with adenosine showed a similar pattern of increasing metabolic activity up to 7 days under hypoxia, but this resulted in eventual decrease in cellular activity after day 7. Based on the microscopic observations of these cell stained with Giemsa, it was not seen that whole population of cells completely reached the state of necrosis up to the latest time point tested, as a few populations of attached cells were still observed. It is expected that those remaining cells eventually die. However, the cells that were supplied with adenosine under the hypoxic conditions maintained a steady state of cellular activity, and these cells resumed their normal metabolic activity two days after adenosine was removed at day 7.

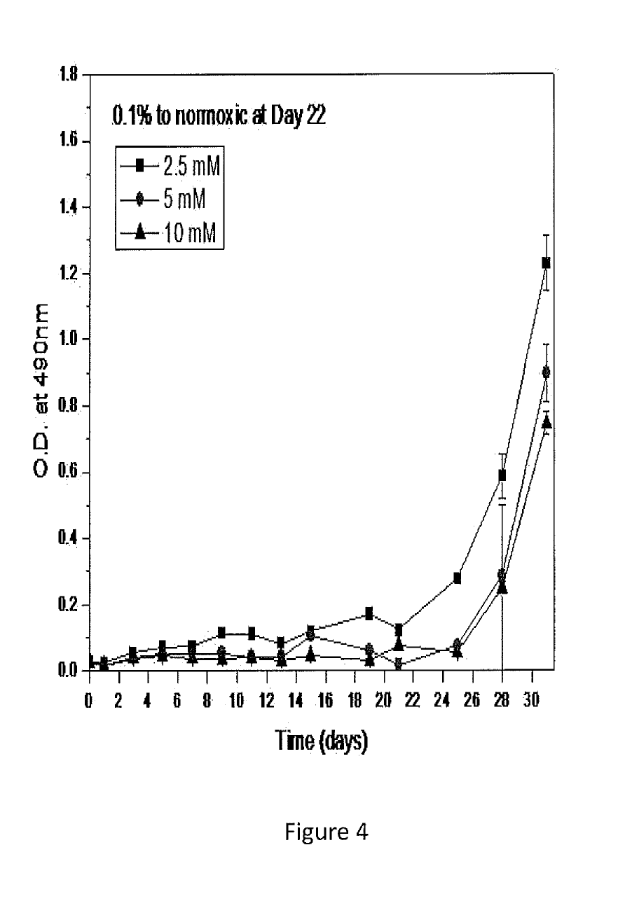

The effect of dose was also evaluated on a degree of cellular activity (FIG. 3). As the dose of adenosine increased from 1 to 10 mM, an escalation of steady hypometabolic state was maintained under hypoxic conditions, and as shown in FIG. 1, the cells were able to resume their normal metabolic activity after 7 days. The cells treated with 1 mM adenosine, however, showed a similar pattern with the cells grown in the hypoxic condition without supply of adenosine (FIG. 2). Based on this outcome, the minimum effective concentration of adenosine appears to be about 1 mM under this experimental condition. The reason for 1 mM treated cells not declining up to the duration tested may potentially have been the lower starting number of cells. This experiment demonstrated that the effects of adenosine on cellular metabolic activity are dose dependent. Finally, the long term effect of adenosine was tested on resuming its proliferation after its supply is stopped. The duration tested under the effect of adenosine was 22 days, as the angiogenesis process is known to take 2-3 weeks (Cotton, 1996, Trends Biotechnol, 14(5):158-62; Padera et al., 1996, Biomaterials, 17(3):277-84) for completion. As shown in FIG. 4, even after 22 days under the effect of adenosine, the cells applied with various adenosine doses still demonstrated the ability to restore its normal proliferation rate.

As demonstrated herein, cell viability can be maintained by downregulating cellular metabolism under hypoxic conditions. Application of adenosine to cells under hypoxic conditions prolonged survival by decreasing the metabolic activity to a steady hypometabolic state, thus reducing oxygen demand. This concept represents a novel method for increasing cellular survival in tissue-engineered constructs during vasculogenesis.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

We claim:

1. A method of increasing the viability of a cell under a hypoxic condition, comprising contacting the cell with an effective amount of adenosine to reduce the oxygen demand of the cell.

2. The method of claim 1, wherein the effective amount of adenosine downregulates the metabolic rate of the cell.

3. The method of claim 1, wherein contacting the cell with an effective amount of adenosine further results in a steady state of cellular metabolic activity.

4. The method of claim 1, wherein the cell resumes a normal proliferation rate when the adenosine is removed from the cell.

5. The method of claim 1, wherein the effective amount of adenosine is greater than about 1 mM.

6. The method of claim 5, wherein the amount of adenosine is between about 1 mM and about 10 mM.

7. The method of claim 1, wherein the cell is a myoblast.

8. The method of claim 7, wherein the cell is a murine myoblast.

9. The method of claim 7, wherein the cell is a human myoblast.

10. A method of increasing cellular survival in a tissue-engineered construct during vasculogenesis, comprising administering an effective amount of adenosine to the cells in the tissue-engineered construct to downregulate the metabolic rate of the cells until host vascularization is established.

11. The method of claim 10, wherein the effective amount of adenosine is greater than about 1 mM.

12. The method of claim 11, wherein the effective amount of adenosine is between about 1 mM and about 10 mM.

13. A method of prolonging the survival of an implanted cell that is under a hypoxic condition in a host, comprising contacting the cell with an effective amount of adenosine to reduce the oxygen demand of the cell until host neovascularization is achieved.

14. The method of claim 13, wherein the effective amount of adenosine downregulates the metabolic rate of the cell.

15. The method of claim 13, wherein contacting the cell with an effective amount of adenosine further results in a steady state of cellular metabolic activity.

16. The method of claim 13, wherein the hypoxic cell resumes a normal proliferation rate when the effects of adenosine are removed.

17. The method of claim 13, wherein the effective amount of adenosine is greater than about 1 mM.

18. The method of claim 17, wherein the amount of adenosine is between about 1 mM and about 10 mM.

19. The method of claim 13, wherein the cell is a myoblast.

20. The method of claim 19, wherein the cell is a murine myoblast.

21. The method of claim 19, wherein the cell is a human myoblast.
